(19) United States
(12) Patent Application Publication
(10) Pub. No.: US 2019/0009973 A1
(43) Pub. Date: Jan. 10, 2019

(54) Canister Device for Producing Sclerosing Foam

(71) Applicant: Provensis Limited, London (GB)

(72) Inventor: David Dakin Iorwerth WRIGHT, London (GB)

(21) Appl. No.: 16/027,724

(22) Filed: Jul. 5, 2018

(30) Foreign Application Priority Data
Jul. 6, 2017 (GB) 1710885.3

(51) Int. Cl. B65D 83/00; B65D 83/48; B65D 83/20; B05B 11/02

(52) U.S. Cl. CPC B65D 83/0061; B65D 83/48; A61K 9/12; B05B 11/02; B65D 83/207

(57) ABSTRACT

The present invention relates to a canister device that is suitable for producing sclerosing foam for intravenous injection, in the treatment of varicose veins and other venous disorders. The canister device for producing therapeutic foam, the device comprises a pressurisable chamber containing sclerosant liquid and physiologically acceptable gas; a foam pathway through which the liquid and the gas may pass from the chamber to the exterior of the device, the pathway including a gas-liquid interface at which the liquid and the gas are mixed and foam is formed; and a mechanism by which the pathway can be opened or closed, such that, when the chamber is pressurised and the pathway is open, the liquid and the gas are forced to pass along the pathway to the exterior of the device; characterised in that an expandable compartment is located within the chamber, the compartment comprising an internal space that is in fluid communication with the exterior of the device through a fluid inlet located on the exterior of the device and that is not in fluid communication with the liquid or the gas, wherein in use fluid is introduced through the fluid inlet to expand the compartment and pressurize the chamber. The device is only pressurized at the point of use and therefore avoid problems associated with shipment and storage of pressurized medical grade gases.

The present invention relates to a device for producing sclerosing foam suitable for intravenous administration. The invention is particularly suitable for producing sterile, clinical grade sclerosing foam for the treatment of varicose veins and venous disorders.

Intravenous administration of sclerosing foam is widely used in the treatment of varicose veins. Traditional devices and methods require preparation (“compounding”) of foam by a physician immediately prior to or even during treatment. As a result, the manual mixing of sclerosing solution and gas tends to produce foam of variable quality, depending on the skills and experience of the compounder. Aerosol-type canisters have been used to address this variability. However, traditional aerosol accelerants such as isopropane may not be used

WO 00/72821 discloses an aerosol-type canister containing a solution of sclerosing agent and a physiologically acceptable gas. The canister is pressurized with the physiologically acceptable gas. When the canister valve is opened, the pressure of the gas provides the force necessary to drive liquid and gas components through a series of passageways, where they are mixed to form foam. Thus, the system produces foam using only the liquid and gas components of the foam product i.e. the aerosol canister operates without the need for traditional aerosol accelerants, such as isopropane, as the accelerant would be incorporated into the foam product and would not suitable for intravenous injection.

WO 02/41872 discloses a similar arrangement but which utilizes two canisters: a first canister containing a solution of a sclerosing agent; and a second canister containing the pressurized physiologically acceptable gas. The pressurized gas is discharged into the first canister to produce the pressurized gas mixture at the point of use. The separation of components into two canisters enables sterilization of each canister without the risk of unwanted degradation of the contents. Foam produced by these canister products has been demonstrated to be effective in the treatment of varicose veins and is approved for use in human patients in the USA and Canada. In practice, however, this system requires the storage and shipment of compressed pharmaceutical grade O₂. It is well understood that all pressurized canisters will leak gas and lose pressure over time and this impacts the shelf-life of the product. It is also understood that the shipment of pressurized oxygen is subject to certain safety provisions, for example, pressurized oxygen may not be transported by air in the United States and, therefore, distribution can be slow, complicated and expensive.

Accordingly, there remains a need for a device for producing foam of the same quality as that which is approved for human use but which does not require significant volumes of pressurized gas. Avoiding the use of pressurized containers will enable longer shelf-life and avoid the distribution issues outlined above.

The present invention provides such a canister. The canister has all the functional features of the canisters described in WO 00/72821 and WO 02/41872 but is adapted to allow pressurization at the point of use using any source of fluid. The fluid used to pressurize the canister is retained separately from the canister contents and is therefore not incorporated into the foam produced by the canister. This removes the need to provide a charging canister containing compressed pharmaceutical grade O₂ (or other physiologically acceptable gas that may be incorporated directly into the foam product). Since the pressurization fluid does not form part of the pharmaceutical product, it need not be supplied by the manufacturer of the product, can be easily incorporated by the user using resources which are readily available in the clinic, such as liquids, compressed air or gas. Distribution and storage of the pharmaceutical product are therefore simpler and cheaper, and shelf life is likely to be increased as leaking of a canister is not a concern.

Accordingly, in a first aspect the present invention provides a canister device for producing therapeutic foam, the device comprising:
a chamber containing sclerosant liquid and physiologically acceptable gas;
a foam pathway through which the liquid and the gas can pass from the chamber to the exterior of the device, the pathway including a foam generating structure; and
a mechanism by which the pathway can be opened to permit passage of the gas and the liquid or closed to prevent passage of the gas and the liquid;
   characterized in that the chamber comprises an expandable inner container in fluid communication with the exterior of the device via an inlet that is not in fluid communication with the foam pathway.

In use, the expandable inner container is filled with a fluid, typically a gas or gas mixture, such that it expands and reduces the space available to the sclerosant liquid and the physiologically acceptable gas within the chamber. The consequent increase in pressure within the chamber is sufficient to force the liquid and the gas through the foam pathway and to form foam. This allows the production of therapeutic foam without the need for a canister system containing pressurized physiologically acceptable gas.

The pressure necessary to produce foam is not provided by an increased pressure of the physiologically acceptable gas which is held in the chamber. Instead, introduction of fluid into an expandable compartment in the chamber serves to pressurize the contents of the chamber. The expandable inner container is sealed to the rest of the canister device, and is therefore formed of a gas-impermeable material. Fluid is introduced to the inner container via an inlet or valve that is able to withstand high pressures to ensure accurate filling and retention of the contents. Since the contents of the expandable inner container do not contact any of the pharmaceutical components present in the canister chamber, the fluid used to pressurize the chamber cannot become incorporated into foam produced by the device. Therefore, fluid introduced into the expandable inner container can be obtained from any source—it need not be of pharmaceutical grade, and it need not be pressurized to a large extent. This eliminates the need for a charging canister containing compressed pharmaceutical grade O₂.

The chamber is a rigid structure, typical of those used in traditional aerosol canisters, that has an outer structure which is impermeable to the sclerosant liquid and the physiologically acceptable gas retained therein. It is capable of withstanding internal pressures significantly greater than those required to force the liquid and the gas through the foam pathway to produce foam. Such pressures are in the range 800 mbar to 4.5 bar gauge (1.8 bar to 5.5 bar absolute), while pressures in the range 1 bar to 2.5 bar gauge are particularly effective for producing foam. Typically chamber should be able to tolerate pressure of 10 bar gauge or even greater. The chamber can be a cylinder, such as a cylinder used to store compressed gases, or it can be a canister, such as a typical aerosol canister. An aerosol canister is advantageous because it is light and inexpensive, and it can be easily modified to introduce atypical features as required.

A sclerosant liquid is an aqueous solution of an irritant substance that causes a localised inflammatory reaction, favouring the elimination of abnormal veins in sclerotherapy. Sclerosant liquids include, e.g. a 1% aqueous solution of polidocanol, but other concentrations of polidocanol are possible, e.g. 0.25-5%, 0.25-1% or 1-5%, and other sclerosing agents include, e.g. sodium tetradecyl sulfate, ethanolamine oleate, sodium morrhuate, hypertonic glucosated or glucosaline solutions, glycerol, chromated glycerol or iodinated solutions. The concentration of sclerosing agent in therapeutic foam can be varied by the user according to the indication to be treated. High concentrations of sclerosing agent (e.g. 1-5% polidocanol) have been reported used in the treatment of large venous malformations whereas lower concentrations of sclerosing agents (e.g. 0.25-1% polidocanol) are typically used in the treatment of spider and reticular veins,

A physiologically acceptable gas is a gas which may be substantially completely (i.e. more than 95%, preferably more than 99%) dissolved in or in other ways absorbed by the blood in a short period, i.e. less than 12 hours, preferably less than 1 hour. Examples of physiologically acceptable gases include oxygen, carbon dioxide, helium and mixtures thereof.

The foam pathway extends from inside the chamber to the exterior of the device, and it provides a conduit through which gas and liquid are delivered under pressure to the foam generating structure and a conduit through which foam is delivered from the foam generating structure to the exterior of the device for administration to a patient. Typically the foam pathway will comprise a dip-tube as typically found in an aerosol canister. The foam pathway will include one or more liquid inlets and one or more gas inlets for the liquid and the gas components that are to be mixed to form foam. The liquid inlet is found at an end of the pathway, typically at the end of a dip-tube, arranged beneath the surface of the liquid in the chamber. The gas inlet is located above the surface of the liquid in the chamber, and it may consist of a hole drilled in a dip-tube. The pathway further comprises one or more outlet orifices through which the foam can pass from the chamber to the exterior of the device.

The foam generating structure provides a means for mixing of gas and liquid by disrupting and restricting the flow of each within the continuous pathway. Typically the gas-liquid interface will be arranged within the continuous pathway such that substantially all of the contents of the pathway are forced through it as they are circulated within the pathway. Preferably, the gas-liquid interface is arranged such that all of the contents of the continuous pathway must pass through it to complete a circuit of the pathway. The gas-liquid interface can comprise an element defining at least one passage of cross sectional area 1 μm² to 10 mm², preferably 10 μm² to 5 mm², more preferably 50 μm² to 2 mm², through which gas and liquid pass when they are propelled through the pathway. The maximum dimension of the passage or passages is preferably between 0.1 μm and 2 mm, more preferably between 1 μm and 1 mm, more preferably between 2 μm and 500 μ, still more preferably between 3 μm an 100 μm. The passage or passages is/are preferably provided by at least one element comprising one or more meshes, screens or sinters. Two or more elements are preferably provided, at least two of the said elements optionally being spaced apart in the direction of flow by between 0.1 mm and 10 mm, preferably between 0.5 mm and 5 mm. In one particular embodiment the gas-liquid interface comprises a mesh stack shuttle that mounts four Nylon 66 meshes held in high density polyethylene (HDPE) rings within an open-ended polypropylene casing. Such meshes typically have a diameter of 6 mm and have a 14% open area made up of 20 μm pores, with the meshes spaced 3.5 mm apart.

The mechanism by which the pathway can be opened or closed is a standard mechanical valve that opens the foam pathway, for example a standard aerosol valve can he crimped into the top of an aerosol canister to serve as such a mechanism. Such a valve is actuated by depressing it to open the pathway and allow its contents to be delivered to the one or more outlet orifices of the pathway.

The canister chamber, the sclerosing agent, the physiologically acceptable gas, the foam pathway, the foam generating structure and the valve are all substantially the same as those described in WO 00/72821 and WO 02/41872, the content of which are hereby incorporated by reference.

The expandable inner compartment comprises an internal space with boundaries that are defined by one or more walls that are made of a material suitable for medical device applications i.e. a material which is sterile or can be sterilized, at least on its outer surface, that is the surface which is contact which the pharmaceutical components (the sclerosing agent and the physiologically acceptable gas). The material is impermeable to the sclerosant liquid and the physiologically acceptable gas in the chamber such that the pressurizing fluid retained in the inner compartment can never contact the pharmaceutical components. The expandable compartment has one or more flexible walls. This allows the compartment to expand easily on introduction of fluid so that the volume occupied by the compartment increases, thereby pressurizing the chamber and its contents. Optionally, the expandable compartment is at least partly made of an elastic membrane. This allows the compartment to be stretched so as to provide higher pressure within the chamber, and it allows the compartment to resume its original volume when deflated thereby allowing the device to be stored for extended periods at lower pressures between uses. Materials suitable for construction of the expandable compartment include silicone, poly vinyl chloride (PVC), polyethylene terephthalate (PET), polyesters and polyurethanes. The materials used to construct the extendable compartment must be inert with respect to the foam being produced and to its component liquid and gas.

The device is typically provided with the internal space of the compartment empty or partially empty, i.e. evacuated or partially evacuated. This allows more space within the chamber for introduction of liquid and gas or for storage and distribution of the device having even lower pressures within the chamber.

The inner compartment may be filled with any fluid that can expand its volume sufficiently to pressurize the contents of the chamber, i.e. liquid or gas. The fluid need not be sterile or of any particular pharmaceutical or industrial grade. This reduces the cost and complexity of using the device as expensive materials are not required for its operation.

The inner compartment is filled (and evacuated) through a valve which is present on the exterior surface of the canister device and provides a gas-tight seal between the exterior surface of the device and the internal volume of the compartment. The valve is preferably one which is able to withstand significantly high pressures (e.g. in excess of 10 bar gauge) to ensure retention of contents without significant leakage. The valve may conveniently be adapted for direct connection to source of fluid, such as a compressed gas cylinder or even an air pump such that a gas tight connection can be easily formed between the valve (or a valve housing) and the source of fluid and, once filled to desired volume/pressure, the source removed without leakage of the contents.

Preferably the valve comprises or vent or release mechanism to allow for release of the contents, such that the device may be stored in its depressurized state. In this regard the canister device can be used to provide more than one dose of foam. In this embodiment, the canister is charged with sufficient sclerosing agent solution and physiologically acceptable gas to produce enough foam to treat more than one patient but pressurized on depressurized between each patient/treatment to minimizes leakage during storage. In such an embodiment the canister is filled with sufficient pharmaceutical contents to produce between 15 and 180 ml of foam.

The canister is typically provided with its contents in an unpressurised state, e.g., at atmospheric pressure, or within 200 mbar of atmospheric pressure such that ingress of contaminants from the external environment is prevented. Optionally, the canister can be pressurized to between 1 and 2 bar absolute. Preferably the canister contents are provided at a pressured of 1.5 bar absolute. Pressurization of the canister is advantageous as it reduces the volume of the canister and can thereby overcome restrictions on volumes that can be shipped, and it also provides a positive pressure to prevent ingress of contaminants into the device.

The fluid used to fill the container may be a liquid or a gas. Using liquid to fill the container is advantageous as it permits the user to achieve a desired pressure by introducing a known volume of liquid because liquids are essentially incompressible. This is particularly useful where the canister is to be used once to deliver a single treatment or a single aliquot of foam. Using gas to fill the container is advantageous because it allows much higher pressures to be achieved due to gases being compressible under pressure. As a consequence of gas being compressible it is possible to achieve higher pressures within the device, and elevated pressure is maintained as foam is produced and the volume of the chamber is reduced. This is particularly advantageous where the device is required for multiple uses or for production of multiple aliquots of foam. Any gas can be used to inflate the inner container provided that the container is impervious to it. Use of air to inflate the container is advantageous as it is readily available and can be easily pressurized using simple pumps or compressors.

The inlet provides a means for introduction of a fluid into the inflatable container. The inlet can be adapted to engage a source of liquid or a source of gas. Optionally the inlet engages a source of gas such as a pump or a compressed gas container and includes a luer connection or a threaded pump engagement. This is advantageous as it provides the same advantages as described above for using gas to inflate the container. Additionally, the inlet can include a one way valve that prevents escape of fluid from the inflatable container after disengagement of the inlet from a source of fluid. This is advantageous as it permits the user to manipulate and move the device without any cumbersome attachments.

Optionally, the device is provided with a vent that permits deflation of the inner container. This allows the user to store the device in an unpressurised state between uses.

The vent can optionally be incorporated into the inlet such that a single structure on the surface of the device provides for introduction of fluid into and evacuation of fluid form the inflatable container.

The device can also include a pressure indicator to indicate to the user when a desired pressure is achieved within the device. This permits the user to monitor pressure and add further fluid as necessary to maintain a desired pressure, and this ensures that consistent foam production is achieved. The pressure indicator can be a simple indicator that provides notification when a desired pressure is reached. This is preferred as it provides valuable information to the user in a low cost and technically simple arrangement. Optionally the pressure indicator can be a pressure gauge that provides a pressure reading to the user. This permits the user to adjust the foam characteristics achieved using the device, and this provides additional flexibility to the user.

Further features and advantages of the invention will be apparent from the following description of specific embodiments, which is made with reference to the accompanying drawings.

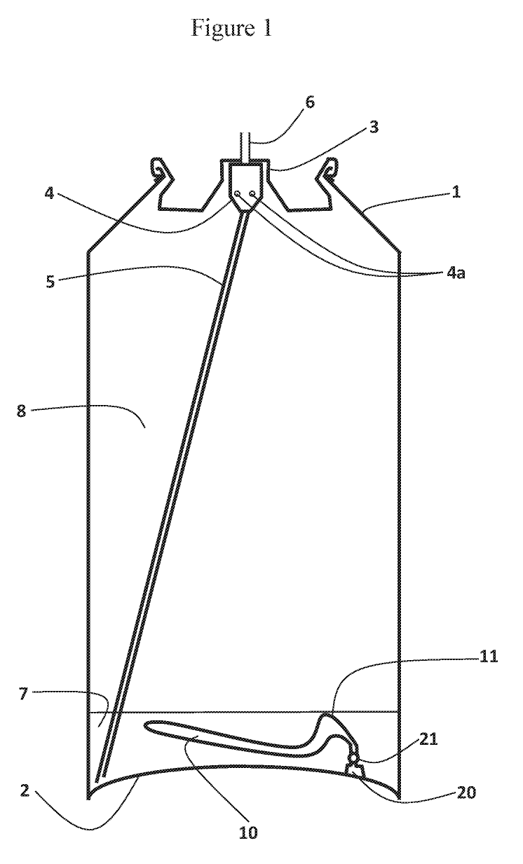

FIG. 1 shows a schematic view of an embodiment of the invention with the inner container in an empty conformation.

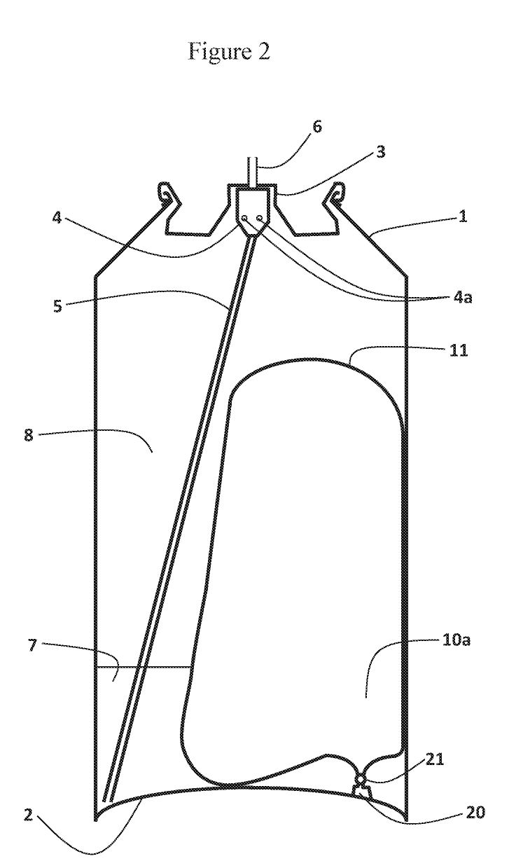

FIG. 2 shows a schematic view of an embodiment of the invention with the inner container in a filled conformation.

A device of the invention is shown in FIG. 1 having a canister body [1] with an inwardly domed bottom surface [2] and having an aerosol valve cup [3] clinched at its top. The canister contains a solution of 1% (v/v) polidocanol [7] and a volume of physiological gas [8] equal to seven times the volume of sclerosant liquid. Within the canister is provided an inner container [10] that is separated from the sclerosant fluid [7] and the physiological gas [8] by an elastic membrane [11]. The elastic membrane [11] is attached via a one way valve [21] to a fitting [20] that is suitable for gas tight connection to a source of gas (not shown) mounted on the domed bottom surface [2].

The valve [3] comprises a gas-liquid interface [4] which includes holes [4a] that permit entry of gas to the interface [4] and mounts a dip-tube [5] that extends below the surface of the sclerosant fluid [7] to allow it to enter the gas-liquid interface. The valve [3] also includes a stem valve [6] that is depressed to activate the aerosol valve [3] and open the foam pathway to the external atmosphere.

In use, the inner chamber [10] is filled by attaching a source of gas (not shown) to the fitting [20] and introducing a volume of air through the one way valve [21]. As shown in FIG. 2, the elastic membrane [11] stretches as the inner chamber [10] is inflated with air. The inflated inner chamber [10a] reduces the available volume in the canister and thereby increases the pressure of the physiological gas [8]. The one way valve [20] prevents air from exiting the inflated inner chamber [10a].

On depression of the valve stem [6], the pressure provided by the inflated inner chamber [10a] forces sclerosant fluid [7] and physiological gas [8] through the foam pathway toward the stem valve [6]. Physiological gas [8] enters the gas-liquid interface [4] through holes [4a] where it mixes with sclerosant fluid [7] that enters via the dip tube [5]. The gas [8] and fluid [7] are forced through the gas-liquid interface under pressure and foam is formed and delivered to a syringe that can be attached directly to the valve stem [6]. Alternatively, a transfer device such as that described in WO 2005/048977 is attached to the valve stem [6] to enable secure, sterile connection of a syringe for dispensing of foam.

1. A canister device for producing therapeutic foam, the device comprising:
a pressurizable chamber containing sclerosant liquid and physiologically acceptable gas;
a form pathway through which the liquid and the gas may pass from the chamber to the exterior of the device, the pathway including a gas-liquid interface at which the liquid and the gas are mixed and the foam is formed; and
a mechanism by which the pathway can be opened or closed, such that, when the chamber is pressurized and the pathway is open, the liquid and the gas are forced to pass along the pathway to the exterior of the device;
characterized in that the expandable compartment is located within the chamber, the compartment comprising an internal space that is in fluid communication with the exterior of the device through a fluid inlet located on the exterior of the device and that is not in fluid communication with the liquid or the gas, wherein in use fluid is introduced through the fluid inlet to expand the compartment and pressurize the chamber.

2. A canister device according to claim 1, wherein the expandable compartment is made at least partly of an elastic membrane selected from silicone, poly vinyl chloride (PVC), polyethylene terephthalate (PET), polyesters and polyurethanes.

3. A canister device according to claim 1, wherein the fluid inlet is adapted to connect with a source of gas.

4. A canister device according to claim 1, wherein the fluid inlet comprises a one way valve.

5. A canister device according to claim 1, wherein the fluid inlet comprises a vent to allow emptying of the expandable compartment.

6. A canister device according to claim 1, further comprising a pressure indicator to inform a user when the chamber is pressurized to a desired level.

7. A canister device according to claim 2, wherein the fluid inlet is adapted to connect with a source of gas.

8. A canister device according to claim 2, wherein the fluid inlet comprises a one way valve.

9. A canister device according to claim 3, wherein the fluid inlet comprises a one way valve.

10. A canister device according to claim 2, wherein the fluid inlet comprises a vent to allow emptying of the expandable compartment.

11. A canister device according to claim 3, wherein the fluid inlet comprises a vent to allow emptying of the expandable compartment.

12. A canister device according to claim 4, wherein the fluid inlet comprises a vent to allow emptying of the expandable compartment.

13. A canister device according to claim 2, further comprising a pressure indicator to inform a user when the chamber is pressurized to a desired level.

14. A canister device according to claim 3, further comprising a pressure indicator to inform a user when the chamber is pressurized to a desired level.

15. A canister device according to claim 4, further comprising a pressure indicator to inform a user when the chamber is pressurized to a desired level.

16. A canister device according to claim 5, further comprising a pressure indicator to inform a user when the chamber is pressurized to a desired level.
